(19) United States
(12) Patent Application Publication
(10) Pub. No.: US 2016/0030120 A1
(43) Pub. Date: Feb. 4, 2016

(54) MANIPULATOR

(71) Applicant: OLYMPUS CORPORATION, Tokyo (JP)

(72) Inventors: Masaru Yanagihara, Tokyo (JP); Kosuke Kishi, Tokyo (JP)

(73) Assignee: OLYMPUS CORPORATION, Tokyo (JP)

(21) Appl. No.: 14/821,170

(22) Filed: Aug. 7, 2015

Related U.S. Application Data
(63) Continuation of application No. PCT/JP2014/053234, filed on Feb. 5, 2014.
(60) Provisional application No. 61/762,369, filed on Feb. 8, 2013.

(51) Int. Cl. A61B 19/00; A61B 1/005; A61B 1/00

(52) U.S. Cl. CPC A61B 19/2203; A61B 1/00133; A61B 1/0055; A61B 2019/2238; A61B 2019/2242; A61B 2019/2211

(57) ABSTRACT

The invention has for its object to provide a manipulator wherein the size of a curved portion is reduced and the internal architecture of the curve portion is simplified. The manipulator 100 comprises an insert part that is capable of being inserted into the body cavity, a movable part that is located at a distal end of the insert part and movable in any direction, a drive unit that is provided on a proximal-end side of the insert part, a first elongated member that is connected to the drive unit and located in the insert part, and displaces in response to operation of the drive unit, a second elongated member that is different from the first elongated member and has a distal end connected to the movable part, and a deceleration mechanism that urges displacement of the second elongated member in association with displacement of the first elongated member, and makes a second amount of displacement of the second elongated member smaller than a first amount of displacement of the first elongated member.

### CROSS-REFERENCES TO RELATED APPLICATIONS

This application is a Continuation Application of International Application PCT/JP2014/053234 filed on Feb. 5, 2014, which claims priority to U.S. Application No. 61/762,369 filed on Feb. 8, 2013. The Contents of International Application PCT/JP2014/053234 and U.S. Patent application No. 61/762,369 are hereby incorporated by reference herein in its entirety.

### BACKGROUND OF THE INVENTION AND RELATED ART STATEMENT

The present invention relates to a manipulator for endoscopes or the like that is inserted into the body cavity and have joints such as curved portions.

So far there has been an endoscope widely available that includes an elongated insert part placed into the body cavity wherein a curved portion at the distal end of the insert part is drawn as by a wire to bend the curved portion thereby viewing the internal organs in the body cavity and applying treatments to them. However, it has been known that because the insert part is of some considerable length, the efficiency of transmission of driving force goes worse, upon transmission of power to the distal-end curved portion, under the influences of friction, wire stretching or elongation, etc. To provide a solution to this problem, for instance, Patent Publication 1 (JP(A) 7-134253) discloses using a flexible shaft as a driving force transmission member and an angle bolt connected to its distal end so that the pitch and direction of the angle bolt are varied to reduce a lowering of transmission efficiency by wire traction thereby varying the angle and direction of bending of the curved portion, etc.

### SUMMARY OF THE INVENTION

The present invention provides a manipulator, comprising:

an insert part that is capable of being inserted into the body cavity,

a movable part that is located at a distal end of the insert part and movable in any direction,

a drive unit that is provided on a proximal-end side of the insert part,

a first elongated member that is connected to the drive unit and located in the insert part, and displaces in response to movement of the drive unit,

a second elongated member that is different from the first elongated member and has a distal end connected to the movable part, and

a deceleration mechanism that urges displacement of the second elongated member in association with displacement of the first elongated member, and makes a second amount of displacement of the second elongated member smaller than a first amount of displacement of the first elongated member.

Preferably, in the manipulator of the invention, the insert part is flexible.

Preferably, in the manipulator of the invention, the second elongated member is shorter than the first elongated member.

Preferably, in the manipulator of the invention, the second elongated member is thinner than the first elongated member.

Preferably, in the manipulator of the invention, the diameter of the movable part for receiving the second elongated member is smaller than the diameter of the insert part for receiving the first elongated member, and the deceleration mechanism is located at a transition portion between the insert part and the movable part at a distal end of the insert part.

Preferably, in the manipulator of the invention, the movable part for receiving the second elongated member is detachable from the insert part for receiving the first elongated member.

Preferably, in the manipulator of the invention, the first elongated member is a first wire with a wire tension adjusting mechanism capable of adjusting the initial tension of the first wire.

Preferably, in the manipulator of the invention, the second elongated member is a second wire with a wire tension adjusting mechanism capable of adjusting the initial tension of the second wire.

Preferably, in the manipulator of the invention, the deceleration mechanism is a pulley drive mechanism.

In the manipulator of the invention, the deceleration mechanism is a rack-and-pinion mechanism.

Preferably, in the manipulator of the invention, the second elongated member is a link formed of a rigid body.

### BRIEF DESCRIPTION OF DRAWINGS

FIG. 1 is illustrative in schematic of the architecture of the endoscope 10 to which the manipulator 100 according to one embodiment of the invention is applied.

FIG. 2 is an enlarged view of the distal-end side of the manipulator 100 according to one embodiment of the invention.

FIG. 3 is a schematic view of the manipulator 100 according to one embodiment of the invention.

FIG. 4 is illustrative in comparison of wire stretching or elongation.

FIG. 5 is an enlarged view of the distal-end side of the manipulator 100 according to another embodiment of the invention.

FIG. 6 is an enlarged view of the distal-end side of the manipulator 100 according to yet another embodiment of the invention.

FIG. 7 is an enlarged view of the distal-end side of the manipulator 100 according to a further embodiment of the invention.

FIG. 8 is illustrative in schematic of the architecture of the endoscope 10 to which the manipulator 100 according to a further embodiment of the invention is applied.

FIG. 9 is illustrative of an example of coupling an insert part to a movable part.

FIG. 10 is illustrative of an example of the deceleration mechanism used with the manipulator 100 according to a further embodiment of the invention.

FIG. 11 is illustrative of an example of the deceleration mechanism used with the manipulator 100 according to a further embodiment of the invention.

FIG. 12 is illustrative of an example of the deceleration mechanism used with the manipulator 100 according to a further embodiment of the invention.

FIG. 13 is illustrative of an exemplary architecture of the master-slave manipulator system.

### DETAILED DESCRIPTION OF THE PREFERRED EMBODIMENTS

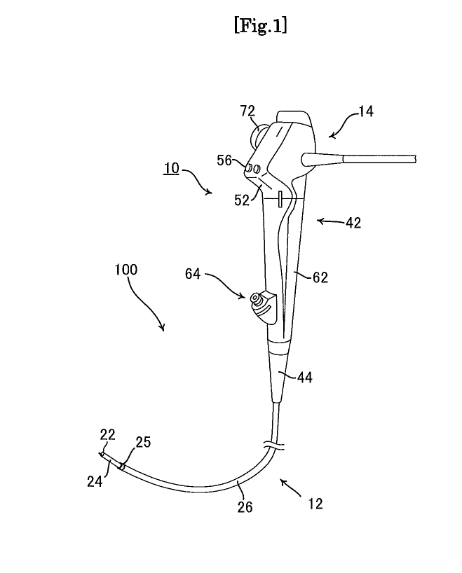

Some embodiments of the invention will now be explained with reference to the accompanying drawings. FIG. 1 is illustrative in schematic of the architecture of the endoscope 10 to which the manipulator 100 according to one embodiment of the invention is applied.

As shown in FIG. 1, the endoscope 10 includes an elongated insert part 12 and a manipulating part 14 located on the proximal-end side of the insert part 12. In order from the distal-end side toward the proximal-end side, the insert part 12 includes a distal-end rigid portion 22, a curved portion 24, a transition portion 25 and a flexible portion 26.

As also shown in FIG. 1, the manipulating part 14 includes a manipulating part body 42 and a bending stopper 44 located at the proximal end of the insert part 12. Positioned below the manipulating part body 42, the bending stopper 44 prevents the flexible portion 26 of the insert part 12 from bending when large force is applied to that flexible portion 26.

The manipulating part body 42 includes a case 52, a curved manipulating portion located such that a portion of a curved manipulating knob (handle) 72 extends out of the case 52, and a plurality of switches 56 for operating a viewing optical system and a lighting optical system. The case 52 is provided with a grip portion 62 gripped by an operator and an opening 64 in the proximal-end side of a forceps channel.

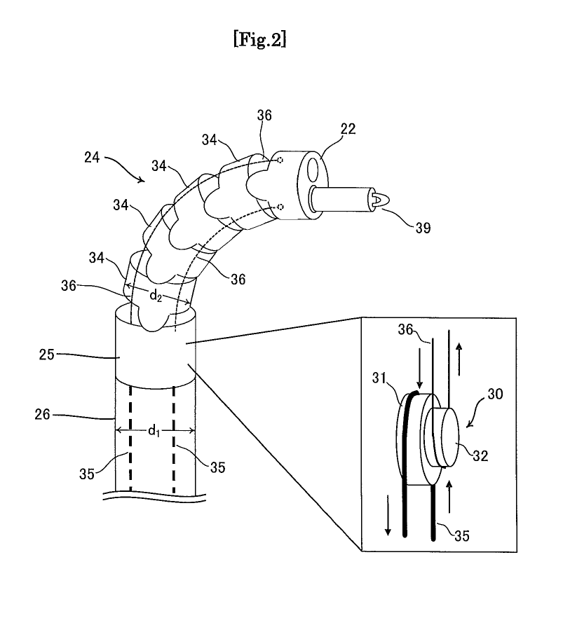

FIG. 2 is an enlarged view of the distal-end side of the manipulator 100 according to the embodiment of the invention here. As shown in FIG. 2, the curved portion 24 is juxtaposed with a plurality of curved pieces 34, each in a substantially ring form, along the axial direction of the curved portion 24. The adjacent curved pieces 34 are capable of rotating relatively. The curved piece 34 adjacent to the distal-end rigid portion 22 is rotatable too.

The most distal-end rigid portion 22 of the curved portion 24 is optionally provided with a treatment tool 39 for surgically cutting off an affected part.

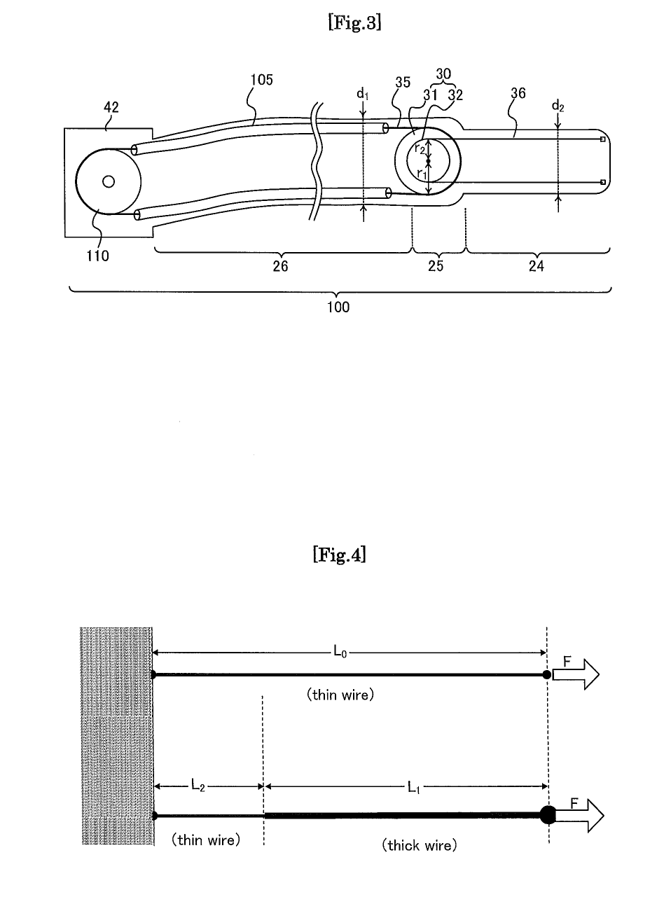

As shown in FIGS. 2 and 3, the distal-end rigid portion 22 is fixedly provided with the distal ends of a pair of second wires 36. In the transition portion 25 located between the curved portion 24 and the flexible portion 26 there is a pulley 30 provided as a deceleration mechanism. The pulley 30 comprises a first diametrical portion 31 (of r1 in radius) and a second diametrical portion 32 (of r2 in radius) smaller in diameter than the first diametrical portion 31. A first wire 35 that is a first elongated member is looped around the first diametrical portion 31 of the pulley 30, and a second wire 36 that is a second elongated member is looped around the second diametrical portion 32. Note here that a path taken by the flexible portion 26 is provided with a coil sheath 105 for keeping a wire path length constant.

The first wire 35 is connected to a pulley for a drive unit 110 moving on the basis of manipulation of the curved manipulating knob 72 in the manipulating part 14, and displaces following movement of the drive unit 110, for instance, in a direction indicated by an action arrow in FIG. 2. Alternatively, the drive unit 110 may be actuated by an actuator such as a motor. In response to this displacement, the pulley 30 rotates, so the second wire 36 displaces too. This in turn drives the curved portion 24 that is a movable part in the manipulator 100 according to the embodiment of the invention here. In the embodiment of the invention here, the pulley 30 used as the deceleration mechanism makes the second amount of displacement of the second wire 36 smaller than the first amount of displacement of the first wire 35 by a deceleration ratio (r2/r1).

Generally, when wires are drawn to drive the curved portion 24, they will be stretched out by traction; however, JP(A) 7-134253, because of being without recourse to any wires, fails to provide any radical solution to a stretching problem arising from the use of wires. According to the deceleration mechanism of the invention, by contrast, the amount of stretching of the first wire 34 caused by traction, too, is reduced by the pulley 30 to r2/r1, thereby easing the flexible portion 26 of the influences of wire stretching and resulting in improvements in precision upon driving of the curved portion 26.

Thus in the manipulator 100 according to the embodiment of the invention here, the pulley 30 in the form of the deceleration mechanism is interposed between the mutually independent first and second wires 35 and 36. According to this manipulator 100, the driving precision of the curved portion 24 can be improved while using the wires as the driving force transmission means for the curved portion 24, and the curved portion 24 can be reduced in size as well.

As is the case with a flexible endoscope having a long flexible portion for transmission of driving force and a short curved portion that is movable upon receipt of driving force, the influences of wire stretching in a flexible portion transmission system are likely to affect precise control of curved portion driving. For the manipulator 100 according to the embodiment of the invention here, it is thus preferable that the second wire 36 is shorter than the first wire 35. If the first and second wires 35 and 36 have the same spring constant, the amount of stretching increases as the length increases, or the shorter the second wire 36 after deceleration, the less the influences of wire stretching become, enabling the distal-end side movable part to follow the necessary tiny movements.

The distal-end curved portion of the insert part 12 is more bendable than the flexible portion, so the second wire 36 that drives the curved portion is more affected by friction (with the curved piece 34 for instance), resulting in the need for increasing the driving force. The use of the deceleration mechanism then allows the drive unit 110 to apply force larger than applied to the first wire 35 to the second wire 36, leading to an additional merit of making the actuator for the drive unit 110 smaller.

For the manipulator 100 according to the embodiment of the invention here, it is preferable that the first wire 35 is thicker than the second wire 36, just only because the first wire 35 that is used a longer distance than the second wire 36 is generally more stretchable; if the wire diameter increases, then influences on wire stretching are reduced to improve transmission, but also because if the second wire 36 is thinner, the distal-end side movable part can be made thinner and more bendable.

Suppose here that to make the manipulator small, a thin wire is used all the way from the proximal-end side manipulating part to the distal-end side movable part. Movement of the movable part will be left affected by wire stretching, causing a problem. However, the first wire 35 being thick and the second wire 36 being thin, as described above, is also effective enough to cope with wire stretching.

FIG. 4 is illustrative in comparison of wire stretching. Note here that FIG. 4 is presented for the purpose of studying a merit of combined use of a thick wire and a thin wire from the standpoint of wire stretching alone, and for the sake of simplification, the deceleration mechanism such as the pulley is left out. Also suppose that the wire is fixed at the left end to a wall or the like.

At the top of FIG. 4, a thin wire is used all over the whole length L₀ from the proximal-end side manipulating part to the distal-end side movable part, and at the bottom of FIG. 4, a thick wire is used a distance L₁ from the proximal-end side manipulating part to the pulley (left out) and a thin wire is used a distance L₂ from the pulley to the movable part.

Suppose here that the thick and thin wires have spring constants k₁ and k₂ and pulled from the right end with force F. At the top of FIG. 4 an elongation Ala may be found from Δ1a=F/(k₂/L₀), and at the bottom of FIG. 4 an elongation Δ1b may be found from

Δ1b=F/(k₁/L₁)+F/(k₂/L₂).

For reference purposes, if L₀=1, L₁=0.8, L₂=0.2, k₁=10, k₂=5 and F=5, then Δ1a=1 and Δ1b=0.6. From this it is found that the combined use of thick and thin wires is smaller in wire elongation than the use of the thin wire all the way, and helps prevent movement of the movable part from being affected by wire stretching.

It is also preferable that the diameter d₂ of the movable part for receiving the second wire 36 (the diameter of the curved pieces 34 of the curved portion 24 in the embodiment of the invention here) is smaller than the diameter d₁ of the insert part 12 for receiving the first wire 35 (the diameter of the tube used for the flexible portion 26 in the embodiment of the invention here), and the deceleration mechanism such as the pulley 30 is located at the transition portion 25 interposed between the insert part 12 and the movable part 25, because the smaller the diameter of the movable part, the better it accommodates itself to a narrow operation space on the distal-end side.

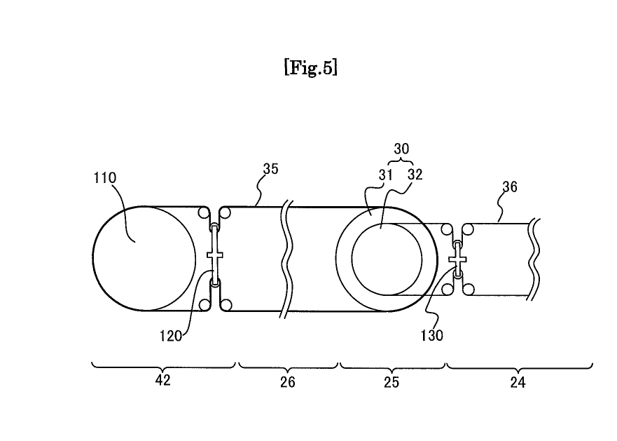

FIG. 5 is an enlarged view of the distal-end side of the manipulator 100 according to another embodiment of the invention.

A pulley 30 is located at the distal end of a flexible portion 26 and the proximal end of a curved portion 24. The pulley 30 comprises a first diametrical portion 31 and a second diametrical portion 32 smaller in diameter than the first diametrical portion 31. The first wire 35 is wound on the first diametrical portion 31 of the pulley 30, and the second wire 36 is wound on the second diametrical portion 32. The second wire 36 has an initial tension adjusted by a second wire tension adjusting mechanism 130, and the first wire 35 has an initial tension adjusted by a first wire tension adjusting mechanism 120. The first wire tension adjusting mechanism 120 is located in the manipulating part body and near the drive unit 110, and the second wire tension adjusting mechanism 130 is located at a transition portion 25. Generally, the curved portion 24 is more bendable than the flexible portion 26 so that the wire passing through the curved portion 24 is more likely to be affected by friction (friction with curved pieces 34 for instance) than the wire passing through the flexible portion 26. If the curved portion 24 is driven through the flexible portion 26 by way of a single wire, then varying frictional forces applied on a path taken by the single wire will cause the tension of the wire to vary, rendering control of wire driving difficult. With the deceleration mechanism according to the embodiment of the invention here, the wire is broken down into two loops and each loop is independently provided with the tension adjusting mechanism, so the tension corresponding to frictional force applied on the first wire 35 and the tension corresponding to frictional force applied on the second wire 36 can be separately adjusted, resulting in the achievement of more precise driving.

Yet another embodiment of the invention will now be explained. In the embodiments so far explained herein, the movable part driven by the second wire 36 is the curved portion 24, whereas in the embodiment of the invention here the movable part driven by the second wire 36 is an arm.

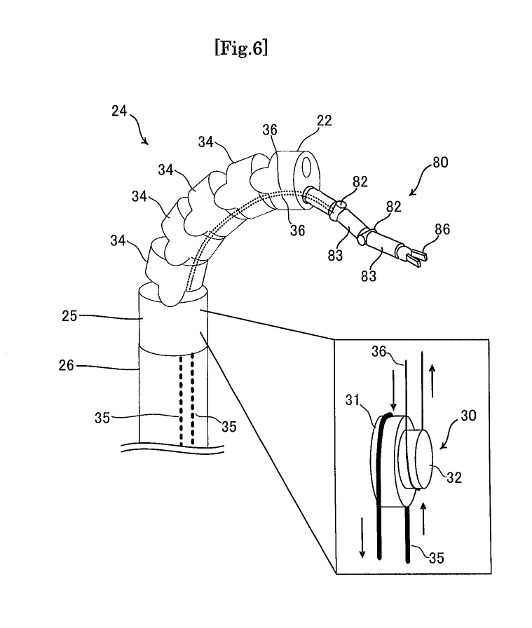

FIG. 6 is an enlarged view of the distal-end side of the manipulator 100 according to this embodiment, wherein an arm 80 is attached to a distal-end rigid portion 22.

The arm 80 includes joint portions 82 that rotate by manipulation of a manipulating rod (not shown), a grip portion 86 attached to the joint portion 86, and a cylindrical portion 83 of continuous length for connecting between the joint portions 82 and connecting the joint portion 82 to the grip portion 86.

In the embodiment of the invention here, a second wire 36 is typically fixed to the joint portions 82, and there is a pulley 30 provided, comprising a first diametrical portion 31 and a second diametrical portion 32. A first wire 35 is wound on the first diametrical portion 31 of the pulley 30, and a second wire 36 is wound on the second diametrical portion 32.

In response to a manipulating rod (not shown), the first wire 35 displaces typically in directions indicated by action arrows in FIG. 6, and the pulley 30 rotates correspondingly, displacing the second wire 36 too. This in turn drives the joint portions 82 that are the movable parts of the manipulator 100. In the embodiment of the invention here, too, the pulley 30 is designed such that the first amount of displacement of the first wire 35 is set larger than that of the second wire 36.

With the manipulator 100 according to such an embodiment of the invention, it is possible to reduce the size of the arm 80 and simplify the internal architecture of the arm 80.

A further embodiment of the invention will now be explained. In the first embodiment of the invention, the movable part that is driven by the second wire 36 is the curved portion 24, but the movable part that is driven by the second wire 36 is a treatment tool 39.

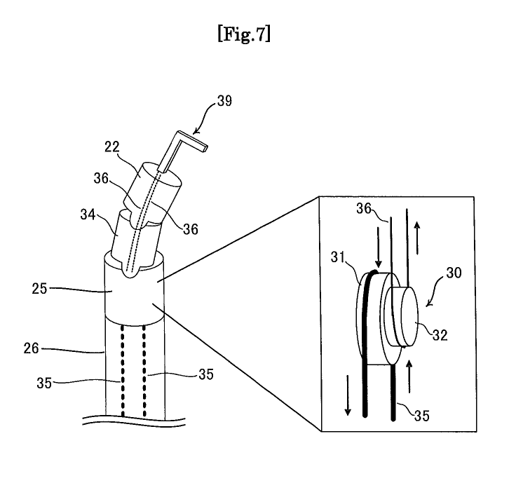

FIG. 7 is an enlarged view of the distal-end side of the manipulator according to the embodiment of the invention here, wherein the treatment tool 39 is attached to a distal-end rigid portion 22.

In the embodiment of the invention here, a second wire 36 is typically fixed to the treatment tool 39, and there is a pulley 30 provided, comprising a first diametrical portion 31 and a second diametrical portion 32. A first wire 35 is wound on the first diametrical portion 31 of the pulley 30, and a second wire 36 is wound on the second diametrical portion 32.

In response to a manipulating rod (not shown), the first wire 35 displaces typically in the directions indicated by action arrows in FIG. 7, and the pulley 30 rotates correspondingly, displacing the second wire 36 too. This in turn drives the treatment tool 39 that is the movable part of the manipulator 100. In the embodiment of the invention here, too, the pulley 30 is designed such that the first amount of displacement of the first wire 35 is set larger than that of the second wire 36.

With the manipulator 100 according to such an embodiment of the invention, it is possible to reduce the size of the distal-end portion and simplify the internal architecture of the distal-end portion.

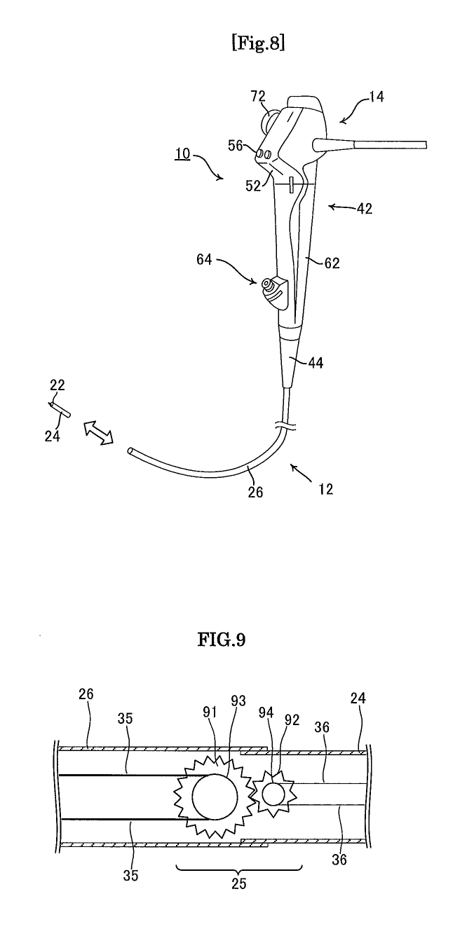

A further embodiment of the invention will now be explained with reference to FIG. 8 that is illustrative in schematic of the internal architecture of an endoscope 10 to which the embodiment of the invention here is applied.

In the first embodiment of the invention, the curved portion 24 for receiving the second wire 36 cannot be detached from the main body. In the embodiment of the invention here, by contrast, the movable part for receiving a second wire 36 (a curved portion 24 herein) is designed in such a way as to be detached from an insert part 12 for receiving the first wire 35 (a flexible portion 26 herein).

According to such an embodiment of the invention, the movable part such as the curved portion 24 can be separated off so that the distal-end portion can be replaced, dispensing with any cleaning of the distal-end side. There is another merit that the distal-end side is transformable depending on the operative method used.

An exemplary mechanism of how to make the movable part for receiving the second wire 36 (the curved portion 24 herein) detachable from the insert part 12 for receiving the first wire 35 (the flexible portion 26 herein) will now be explained with reference to FIG. 9 that is illustrative of an example of coupling the insert part 12 to the movable part.

The flexible portion 26 forming the insert part 12 includes therein a first gear 91 and a first pulley 93 that rotates coaxially with the first gear 91, and there is a first wire 35 wound on the first pulley 93, which wire displaces in response to manipulation of a curved manipulating knob 72 in a manipulating part 14.

The curved portion 24 that is the movable part includes therein a second gear 92, and a second pulley 94 that rotates coaxially with the second gear 92, and there is a second wire 36 wound on the second pulley 94, which wire drives the movable part through its displacement.

The first gear 91 is set larger in diameter than the second gear 92, and the first pulley 93 is set larger in diameter that the second pulley 94.

The insert part 12 (the flexible portion 26 herein) is coupled to the movable part (the curved portion 24 herein) to mate the first gear 91 with the second gear 92 thereby transmitting driving force from the first wire 35 to the second wire 36.

A further embodiment of the invention will now be explained. In the embodiments of the invention so far described, for instance and without limitation, the pulley(s) is used as the deceleration mechanism. In what follows, a specific deceleration mechanism usable in the invention will be explained.

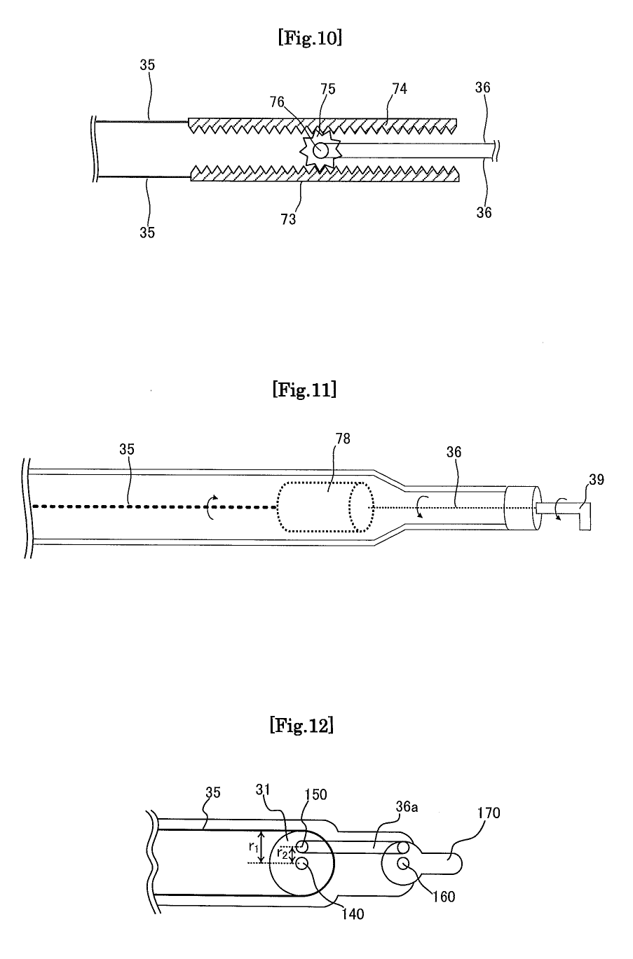

FIG. 10 is illustrative of an exemplary deceleration mechanism used with the manipulator 100 according to a further embodiment of the invention. First and second racks 73 and 74 are located in opposing relations. The first and second racks 73 and 74 are each fixedly provided with a first wire 35 that displaces in response to manipulation of a curved manipulating knob 72 in a manipulating part 14.

There is a pinion 75 provided to mate with the first and second racks 73 and 74. The pinion 75 is provided with a pulley 76 in a coaxial relation thereto, and a second wire 36 that drives the movable part is wound on the pulley 76.

Even with such a deceleration mechanism as described above, it is possible to reduce the second amount of displacement of the second wire 36 relative to the first amount of displacement of the first wire 35 thereby accomplishing the object of the invention. Plus, it is possible to reduce the influences of stretching or elongation of the first wire 35 in the flexible portion 26.

FIG. 11 is illustrative of an exemplary deceleration mechanism used with the manipulator 100 according to a further embodiment of the invention wherein a first wire 35 displaces rotationally, and this rotational displacement is transmitted to a second wire 36 to rotate a treatment tool 39 for instance.

In the embodiment of the invention here, a planetary gear 78 is employed as a mechanism for transmitting the rotational displacement of the first wire 35 to the second wire 36 thereby carrying out deceleration.

Even with the deceleration mechanism with recourse to the planetary gear 78, it is possible to reduce the second amount of displacement of the second wire 36 thereby accomplishing the object of the invention. Plus, it is possible to reduce the influences of elongation of the first wire 35 in the flexible portion 26.

FIG. 12 is illustrative of an exemplary deceleration mechanism used with the manipulator 100 according to a further embodiment of the invention. A first wire 35 is wound up around a first diametrical portion 31 of r1 in radius, and drawn from a drive unit 110. The first diametrical portion 31 swivels by displacement of the first wire 35 with an axis 140 as center. The first diametrical portion 31 is connected with one end of a link 36a via a pin 150 in a position a distance r2 away from the axis 140, and the other end of the link 36a is connected to a joint 170 that swivels with an axis 160 as center, providing a deceleration mechanism where r1>r2. Even with this deceleration mechanism that drives a curved portion 24 with the link, it is possible to reduce the influences of elongation of the first wire 35 in a flexible portion 26.

It is to be noted that each of the embodiments as described above is explained typically with reference to one integral structure of the manipulating and insert parts 14 and 12; however, the invention may also be applied to the so-called master-slave manipulator system wherein the manipulating and insert parts are each remotely controlled.

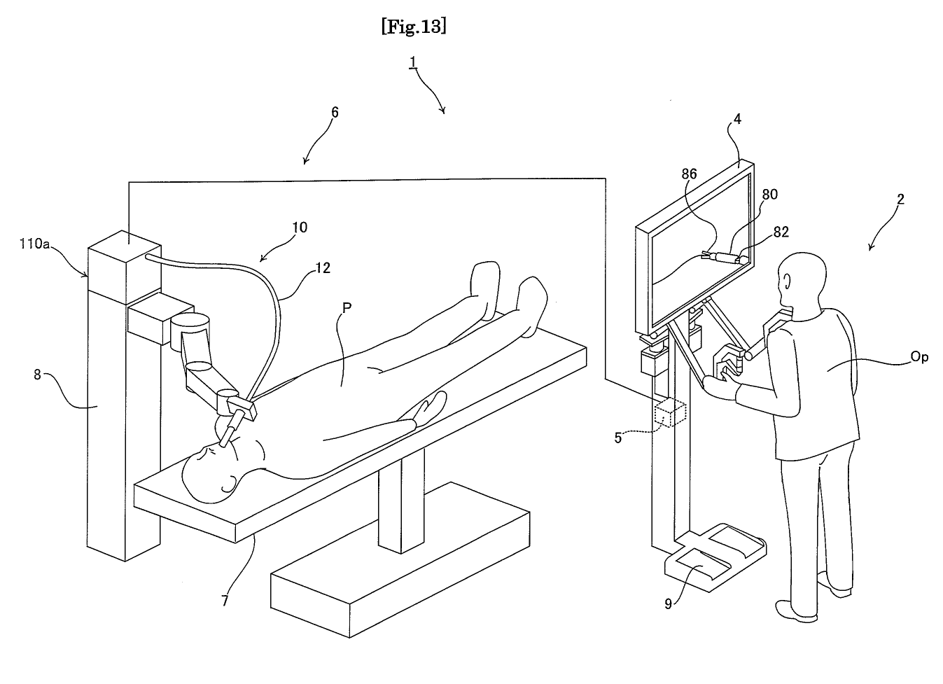

For instance, the invention may be applied to a master-slave manipulator system comprising a master manipulator 2 manipulated by an operator Op and a slave manipulator 6 having the endoscope device 10 typically shown in FIG. 6, as shown in FIG. 13.

The master manipulator 2 comprises a master arm 3 into which an input is entered by the operator Op, a display unit 4 for displaying images taken by the endoscope device 10, etc., a control unit 5 for generating manipulation commands for putting the slave manipulator 6 in operation based on the movement of the master arm 3, and a foot switch 9 for selecting control modes.

In the embodiment of the invention here, the master arm 3 is a manipulating part for moving the respective portions of the slave manipulator 6 including an arm 80 attached to the endoscope device 10. Although not shown in details, the master manipulator 2 includes a pair of master arms corresponding to the right and left hands of the operator Op, respectively. The master arm 3 includes a multi-joint structure, one moving a joint portion 82 of the arm 80 and the other moving a curved portion 24. The master arm 3 is provided at an end where the operator Op takes position with a grip manipulating portion (not shown) for moving a grip portion 86 of the arm 80.

The display unit 4 is provided for displaying images taken of the site being treated by a viewing or lighting optical system or the like attached to the endoscope device 10. Just only the site being treated but also the arm 80 and grip portion 86 are displayed in the display unit 4.

The slave manipulator 6 comprises a table 7 on which a patient P lies down, a multi-joint robot 8 positioned near the table 7, and an endoscope device 10 mounted on the multi-joint robot 8. The multi-joint robot 8 and endoscope device 10 are operated by a drive unit 110A provided on the slave manipulator 6 according to an manipulation command coming from the master manipulator 2, although not shown in details.

As shown in FIG. 13, the patient P is laid down by a assistant (not shown) on the table 7 for proper treatments such as disinfection, anesthetization or the like.

The operator Op inserts the insert part 12 from the mouth of the patient P into the body cavity while giving appropriate instructions to the assistant. Then, the operator Op manipulates the master arm 3 to bend the curved portion 24 of the insert part 12 and move the arm 80 and grip portion 86, as occasion may be, while checking up the images on the display unit 4.

Even when the invention is applied to such a master-slave manipulator system as described above, it is possible to obtain the advantages described with reference to the aforesaid embodiments of the invention.

In some embodiments of the invention as described above, the wire(s) is described as being looped around the pulley; however, the wire may be wound on the pulley while fixed at the end to the pulley.

It is to be noted that just only the wire or link but also a flexible shaft explained with reference to the embodiment of FIG. 11 may be used as the elongated member.

### REFERENCE SIGNS LIST

2: master manipulator
3: master arm
4: display unit
5: control unit
6: slave manipulator
7: table
8: multi-joint robot
9: foot switch
10: endoscope (endoscope device)
12: insert part
14: manipulating part
22: distal-end rigid portion,
24: curved portion
25: transition portion
26: flexible portion
30: pulley
31: first diametrical portion
32: second diametrical portion
34: curved piece
35: first wire
36: second wire
36a: link
39: treatment tool
42: manipulating part body
44: bending stopper
52: case
56: switch
62: grip portion
64: distal-end side opening
72: curved manipulating knob (handle)
73: first rack
74: second rack
75: pinion
76: pulley
78: planetary gear
80: arm
82: joint portion
83: cylindrical portion
86: grip portion
91: first gear
92: second gear
93: first pulley
94: second pulley
100: manipulator
105: coil sheath
110: drive unit
110A: drive unit
120: first wire tension adjusting mechanism
130: second wire tension adjusting mechanism
140: axis
150: pin
160: axis
170: joint
Op: operator
P: patient

1. A manipulator, comprising:
an insert part that is capable of being inserted into the body cavity,
a movable part that is located at a distal end of the insert part and movable in any direction,
a drive unit that is provided on a proximal-end side of the insert part,
a first elongated member that is connected to the drive unit and located in the insert part, and displaces in response to movement of the drive unit,
a second elongated member that is different from the first elongated member and has a distal end connected to the movable part, and
a deceleration mechanism that urges displacement of the second elongated member in association with displacement of the first elongated member, and makes a second amount of displacement of the second elongated member smaller than a first amount of displacement of the first elongated member.

2. The manipulator according to claim 1, wherein the insert part is flexible.

3. The manipulator according to claim 1, wherein the second elongated member is shorter than the first elongated member.

4. The manipulator according to claim 1, wherein the second elongated member is thinner than the first elongated member.

5. The manipulator according to claim 1, wherein a diameter of the movable part for receiving the second elongated member is smaller than a diameter of the insert part for receiving the first elongated member, and the deceleration mechanism is located at a transition portion between the insert part and the movable part at a distal end of the insert part.

6. The manipulator according to claim 1, wherein the movable part for receiving the second elongated member is detachable from the insert part for receiving the first elongated member.

7. The manipulator according to claim 1, wherein the first elongated member is a first wire with a wire tension adjusting mechanism capable of adjusting an initial tension of the first wire.

8. The manipulator according to claim 7, wherein the second elongated member is a second wire with a wire tension adjusting mechanism capable of adjusting an initial tension of the second wire.

9. The manipulator according to claim 1, wherein the deceleration mechanism is a pulley drive mechanism.

10. The manipulator according to claim 1, wherein the deceleration mechanism is a rack-and-pinion mechanism.

11. The manipulator according to claim 1, wherein the second elongated member is a link formed of a rigid body.
